(19) United States
(12) Patent Application Publication
(10) Pub. No.: US 2005/0143328 A1
(43) Pub. Date: Jun. 30, 2005

(54) COMPOSITION AND TREATMENT FOR ENVELOPE VIRUS INFECTIONS

(76) Inventor: Philip M Steele, Allen, TX (US)

Correspondence Address: BAKER & DANIELS, 300 NORTH MERIDIAN STREET, SUITE 2700, INDIANAPOLIS, IN, 46204-1782, US

(21) Appl. No.: 10/977,779

(22) Filed: Oct. 29, 2004

Related U.S. Application Data
(63) Continuation of application No. 60/516,369, filed on Oct. 31, 2003.

(51) Int. Cl. A61K 31/704

(52) U.S. Cl. 514/35; 514/37; 514/39

(57) ABSTRACT

Abstract of the Disclosure

The present invention discloses both a compound and a method for treating and preventing the spread of envelope virus infections by using organic molecules, including 2-deoxystreptamine aminoglycoside molecules and their derivatives, to inhibit viral protein translation and viral invasion of cells. Those organic molecules, which are able to bind to RNA and inhibit translation of envelope virus proteins, can be incorporated into a compound comprising a pharmaceutically acceptable carrier and administered in a physiologically appropriate manner to individuals exposed to or infected with envelope viruses. This invention includes, but is not limited to, the treatment of DNA-containing envelope virus infections, such as herpes simplex-1, and amelioration and prevention of the symptoms of such infections, such as herpes-related cold sores.

### Detailed Description of the Invention

### BACKGROUND OF THE INVENTION

This invention relates generally to compositions and compounds comprising organic molecules that bind to RNA and inhibit translation of envelope virus protein, antiviral technology and envelope virus treatment and prevention by utilizing said compositions and compounds. Specifically, the field of the invention relates to antiviral technology, both for preventing envelope virus infections and for ameliorating or preventing the symptoms of envelope virus infection outbreaks in individuals already infected.

### DESCRIPTION OF THE RELATED ART

Aminoglycoside molecules have been shown to inhibit protein translation, self-splicing group I introns, RNase P and small ribozymes in vitro (Schroeder, R, Waldsich, C, and Wank, H (2000) Modulation of RNA function by aminoglycoside antibiotics. EMBO J 19[1]:1-9). In addition, Werstuck et al. disclose using these molecules to control gene expression in vivo using Escherichia coli bacteria (Werstuck, G and Green, MR (1998) Controlling Gene Expression in Living Cells Through Small Molecule-RNA Interactions. Science 282[5387]:296-298), and Zapp et al. disclose that some aminoglycoside compounds are able to be used in vivo to interfere with the binding of RNA and certain elements necessary for HIV-1 production (Zapp, ML, Stern, S, and Green, MR (1993) Small molecules that selectively block RNA binding of HIV-1 Rev protein inhibit Rev function and viral production. Cell 74[6]:969-978). Green et al., U.S. Pat. No. 5,534,408, discloses aminoglycoside use in inhibiting HIV Rev/Rev Response Element binding. Gustafson et al., U.S. Pat. No. 5,942,547, disclose use of certain aminoglycoside derivatives in inhibiting HIV Rev/Rev response Element binding.

Research publications also have disclosed that certain aminoglycoside compounds are able at least partially to inhibit the initial infection of baby hamster kidney cells by the herpes simplex-1 virus (Langeland, N, Haarr, L, and Holmsen, H (1986) Evidence that neomycin inhibits HSV 1 infection of BHK cells. Biochem Biophys Res Commun 141[4]:198-203; Herold, BC and Spear, PG (1994) Neomycin inhibits glycoprotein C (gC)-dependent binding of herpes simplex virus type 1 to cells and also inhibits postbinding events in entry. Virology 203[1]:166-171). Disclosure also has been made for using the binding capabilities of neomycin to try to ascertain which herpes simplex-1 glycoproteins are necessary for adsorption to and infection of baby hamster kidney cells, rabbit skin cells, D-54 human glioma cells, and normal human glia cells (Langeland, N, Oyan, AM, Marsden, HS, Cross, A, Glorioso, JC, Moore, LJ, and Haarr, L (1990) Localization on the herpes simplex virus type 1 genome of a region encoding proteins involved in adsorption to the cellular receptor. J Virol 64[3]:1271-1277). Neomycin also has been used in a study of herpes simplex-1 adsorption to and infection of certain types of oral epithelial cells (Hung, SL, Wang, YH, Chen, HW, Lee, PL, and Chen, YT (2002) Analysis of herpes simplex virus entering into cells of oral origin. Virus Res 86[1-2]:59-69).

Conjugates of aminoglycoside molecules also have been the subject of envelope virus research. In particular, arginine conjugates have been used by both Borkow et al. and Litovchick et al. in studies of anti-HIV activities (Borkow, G, Vijayabaskar, V, Lara, HH, Kalinkovich, A, and Lapidot, A (2003) Structure-activity relationship of neomycin, paromomycin, and neamine-arginine conjugates, targeting HIV-1 gp120-CXCR4 binding step. Antiviral Res 60[3]:181-192; Litovchick, A, Evdokimov, AG, and Lapidot, A (2000) Aminoglycoside-arginine conjugates that bind TAR RNA: synthesis, characterization, and antiviral activity. Biochem 39[11]:2838-2852; Litovchick, A, Lapidot, A, Eisenstein, M, Kalinkovich, A, and Borkow, G (2001) Neomycin B-arginine conjugate, a novel HIV-1 Tat antagonist: synthesis and anti-HIV activities. Biochem 40[51]:15612-15623).

### SUMMARY OF THE INVENTION

This invention involves at least one organic molecule that binds to RNA and inhibits translation of envelope virus proteins. Such antiviral activity will decrease the viral titer in the affected tissues and cells of the host organism, thereby slowing the infection rate and ameliorating the symptoms of viral infection in an already-infected individual. Further, many of the included classes of organic molecules also have the ability to bind to envelope virus proteins (especially when said molecules are conjugated to one or more amino acids like arginine), thus making them useful in the prevention of envelope virus attachment to and penetration of host cells. Also taught in this invention are methods of using those organic molecules as part of a compound that also comprises of a pharmaceutically acceptable carrier, which is administered in a physiologically appropriate manner to prevent an initial infection, slow an existing infectious process, or ameliorate the symptoms of an existing envelope virus infection or viral outbreak episode. This invention helps improve prevention and treatment of envelope virus infections in multicellular organisms, including humans.

Envelope virus infections are common throughout the world, causing illnesses that range from one-time sicknesses like chicken pox to recurring disease outbreaks like herpes simplex-1 to chronic (and sometimes fatal) syndromes like HIV/AIDS. Much of the research in both the scientific and health care communities has focused on the prevention of these infections, either by encouraging individuals to avoid exposure to the viruses or focusing on inhibition of viral invasion into host cells once exposure has occurred. For the millions of people already infected, however, there is a real need for effective treatment of viral symptoms and reduction of systemic viral loads. The present invention addresses a more perplexing problem that past research leaves largely untouched: how to relieve the symptoms of and reduce the viral load present in organisms that have an existing envelope virus infection. This invention can provide millions of humans – and other infected organisms – with an effective treatment to help control the course of envelope virus disease progression. For many affected individuals, this invention can help provide freedom from painful, embarrassing, or debilitating complications and manifestations of envelope virus infections.

This invention utilizes the ability of 2-deoxystreptamine aminoglycoside (hereinafter, "2-DosA") compounds, their derivatives, and other related molecules to bind to RNA and disrupt the process of viral protein translation – and therefore, viral production – in infected cells. The result is a method for treating envelope virus infections that effectively decreases the number of viruses being produced in the host organism, allowing that infected individual to experience relief from and control of virus-related symptoms, including in some instances the prevention of virus-induced symptomatic outbreaks.

### DESCRIPTION OF THE PRESENT INVENTION

The embodiments disclosed below are not intended to be exhaustive or limit the invention to the precise form disclosed in the following detailed description. Rather, the embodiment is chosen and described so that others skilled in the art may utilize its teachings.

This invention teaches a composition comprising at least one organic molecule that binds to RNA and inhibits translation of envelope virus protein, and a pharmaceutically acceptable carrier. It also discloses a method of treating envelope virus infections in multicellular organisms, comprising the steps of administering a composition in a physiologically appropriate manner to the organism infected with an envelope virus, wherein the composition is administered at least one time, wherein the composition comprises at least one organic molecule that binds to RNA and inhibits translation of envelope virus protein and a pharmaceutically acceptable carrier. Additionally, it teaches a method of treating skin lesions caused by at least one envelope virus in multicellular organisms, said method comprising administering a composition in a physiologically appropriate manner, wherein the composition is administered at least one time, wherein the composition comprises at least one organic molecule that binds to RNA and inhibits translation of envelope virus protein and a pharmaceutically acceptable carrier.

Envelope viruses are among the most common pathogens infecting humans; at any given time, millions of people world-wide are suffering from some type of envelope virus infection. Some of the more well-known of these viruses include herpes simplex virus type 1, herpes simplex virus type 2, varicella zoster virus, toga virus, syncytial virus, paramyxovirus, myxovirus, human herpes virus-6, human immunodeficiency virus (HIV), cytomegalovirus, corona virus, hepatitis A, B, C, D, E, and G, Epstein-Barr viruses, etc. Depending on the particular virus and the particular host organism involved, envelope virus infections can be fatal.

Envelope viruses, while a diverse group of pathogens, nevertheless share certain similarities in structure and infection mechanism. These viruses have a central core of either DNA (e.g., herpes simplex-1) or RNA (e.g., HIV). That nucleic acid core is surrounded by a capsid (whose shape may vary from virus to virus), and the nucleic-acid-containing capsid is then "enveloped" in a membrane from which one or more lipid glycoproteins protrude. These glycoproteins are critical to an envelope virus's ability to bind to, and eventually penetrate, the cell membrane of its host.

An envelope virus uses one or more of these glycoproteins to attach to certain receptor(s) that are already present on the host cell (Herold, BC and Spear, PG (1994) Neomycin inhibits glycoprotein C (gC)-dependent binding of herpes simplex virus type 1 to cells and also inhibits postbinding events in entry. Virology 203[1]:166-171). Once this attachment is made, the envelope virus fuses with the cell membrane of its host, effectively injecting its genetic material (either DNA or RNA) into the host cell. The virus then uses the host's enzymes, ribosomes, transfer RNA (tRNA), and adenosine triphosphate (ATP) to replicate its genetic material and transcribe and translate its DNA -- or reverse transcribe, transcribe, and translate its RNA – into proteins needed to reassemble and release new virus particles. In essence, envelope viruses turn their host cells into virus-making machines, and the newly minted viruses go on to infect other cells in the host organism. This invasion, replication, and re-infection process, when it occurs on a large enough scale, is what causes clinical manifestations of envelope virus disease.

The herpes simplex viruses (HSV) provide a well-known example of a typical envelope virus infection. HSV initially uses glycoproteins on its envelope membrane to bind to host neurons' receptor molecules. Such binding permits the HSV to fuse to the host cell and inject its genetic material into the dendritic area of the neuron, from whence it travels back up to the cell body of the neuron. After the primary infection occurs, the herpes simplex virus maintains a latent infection in the neurons of the host organism's sensory ganglia (for HSV-1, the trigeminal ganglion; for HSV-2 the sacral ganglia) and occasionally causes eruptions of infectious blisters (for HSV-1, in the orofacial region; for HSV-2 in the genital region) and skin-related symptoms including inflammation, redness, tingling, swelling, itching, burning, cracking, blistering, bleeding, oozing, or weeping of the skin and related regions. The neurons maintain a reservoir of herpes viruses, which allows the infections and blister eruptions to recur when triggered by stress, hormone fluctuations, exposure to sunlight, and many other factors. The herpes simplex virus can be spread to others even if the host organism is asymptomatic (i.e. no one has diagnosed, or noticed, any of the clinical symptoms related to the existence of infected cells, such as presence of viral antibodies in body fluids, diagnostic levels of viral titer in body fluids, pain swelling, burning, inflammation, redness, tingling, itching, skin lesions, etc.) or is between clinically evident infection episodes. In some instances, serious diseases such as neonatal disseminated herpes, viral encephalitis, or blinding keratitis can result. Immuno-compromised individuals are more likely to experience frequent and severe clinically evident infection episodes.

Herpes simplex viruses, like the other envelope viruses, are still dependent on the host cell not only for receptor proteins to which they can attach, but also for the raw materials they need to replicate, repackage, and disseminate themselves throughout the host organism. Originally, scientists believed that viruses in general were unresponsive to any kind of antibiotic therapy. In the fight against envelope viruses, however, researchers recently have found organic compounds that are thought to inhibit both the process of envelope virus attachment and the process of envelope virus protein translation. These compounds include the aminoglycoside molecules (including streptamine-containing aminoglycoside molecules, spectinamine-containing aminoglycoside molecules, and especially 2-DosA compounds and their derivatives) and certain other related molecules, like viomycin (of the tuberactinomycin family of compounds). While early attempts at discerning and exploiting antiviral compounds centered around the use of antisense molecules targeted at key RNA targets (see, e.g., Branch, AD (1998) A good antisense molecule is hard to find. Trends Biochem Sci 23[2]:45-50), the ostensible ability of aminoglycoside and related molecules to inhibit viral protein translation by interacting with RNA structural motifs also has been noted (e.g., hairpin loops, internal loops, nucleic acid bulges, or the major groove of double-stranded RNA) (see, e.g., Fourmy, D, Recht, MI, Blanchard, SC, and Puglisi, JD (1996) Structure of the A site of Escherichia coli 16S ribosomal RNA complexed with an aminoglycoside antibiotic. Science 274[5291]:1367-1371).

### AMINOGLYCOSIDE (AND RELATED) MOLECULES AND POSSIBLE ANTIVIRAL MODES OF ACTION

The invention involves the use of aminoglycoside molecules to inhibit the attachment, penetration, and replication of envelope viruses. The aminoglycoside molecules suggested for use in this invention include, but are not limited to, those containing streptamine (e.g., streptomycin), spectinamine (e.g., spectinomycin), and 2-deoxystreptamine (e.g., amikacin, butirosin, geneticin, gentamicin C1, gentamicin C1a, gentamicin C2, lividomycin, lividomycin A, neamine, neomycin, neomycin B, netilmicin, paromomycin, ribostamycin, sisomycin, tobramycin) bases.

Aminoglycoside molecules generally either are found as natural products of soil actinomycete (and often secreted as part of a mixture of multiple related molecules), or are derived from such products. (Indeed. several aminoglycoside derivatives have been created by scientists to date (e.g., Gustafson, GR, Powers, DG, and Wuonola, MA. Use of 2-deoxystreptamine as a molecular scaffold for the preparation of functionally and spatially diverse molecules and pharmaceutical compositions thereof. Scriptgen Pharmaceuticals, Inc. 273964[6140361]. 10-31-2000. Waltham, MA. 3-22-1999)). At biological pH, aminoglycoside molecules usually exist as cations. They may be functionalized by various modifying groups (e.g. H, OH, NH₂, mannosyl, NHCH₃, etc.), whose identities, presence, or absence serve to distinguish the various derivatives, analogs, and conjugates of these aminoglycoside molecules from one another and make them more or less useful for certain embodiments of the invention. These organic molecules generally are constructed around a pharmacophoric 1,3-diaminocyclohexane unit (as illustrated by Structure I below), and generally are created according to the following pattern (with the different combinations of R groups providing the structures of the different aminoglycoside molecules and their derivatives, analogs, and conjugates): Structure I: 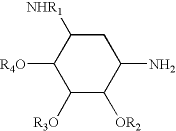 , wherein R₁ comprises H, CH₂CH₃, or L-(-)-4-amino-2-hydroxybutyrl (additionally recognizing that either NH₂, NHR₁, or both may be independently substituted with an arginine); wherein R₂ comprises of 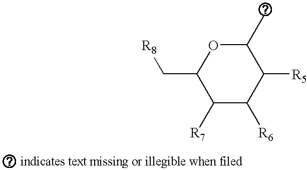 , 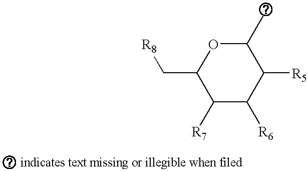 , or 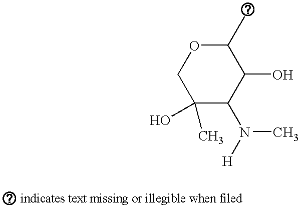 ; wherein R₅ comprises NH₂, OH, or arginine; wherein R₆ comprises H or OH; wherein R₇ comprises H or OH; wherein R₈ comprises NH₂, OH, or arginine; wherein R₉ comprises H or OH; wherein R₁₀ comprises H or OH; wherein R₁₁ comprises NH₂, OH, NHCH₃, or arginine; wherein R₃ comprises H, 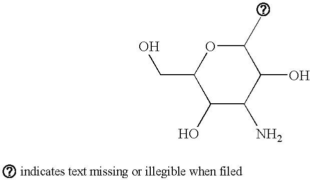 , 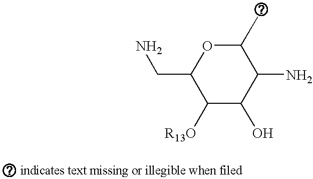 , 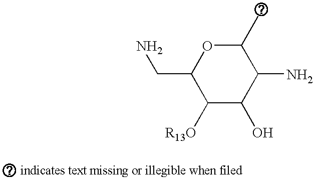 , or 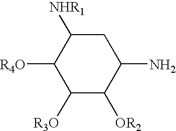 (recognizing that either or both of the amine groups in 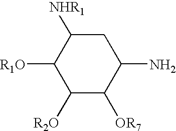 may be substituted with arginine); wherein R₁₂ comprises H or 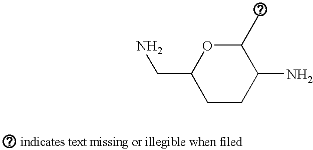 (recognizing that either or both of the amine groups in 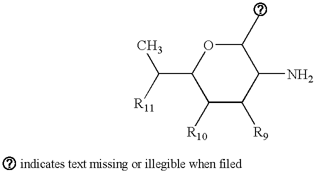 may be independently substituted with arginine); wherein R₁₃ comprises H, mannosyl, 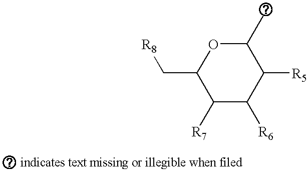 , 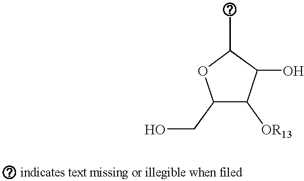 , or 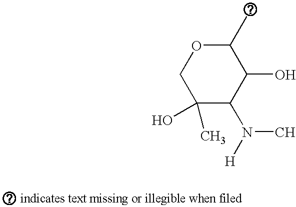 ; and wherein R₄ comprises H, 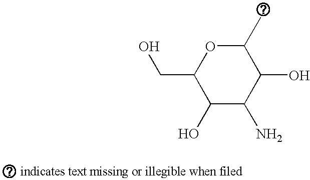 , 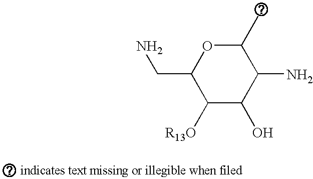 , 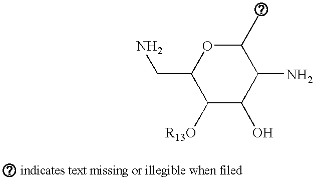 , or 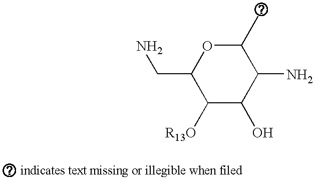 (recognizing that either or both of the amine groups in 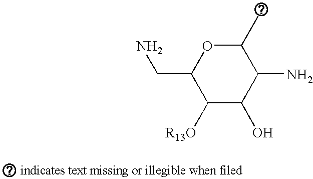 may be independently substituted with arginine).

The manner of synthesis of aminoglycoside molecules is well-known to those skilled in the art. The stable structure of aminoglycoside molecules, as well as their easily interchangeable functional groups, has led scientists to make several derivatives, conjugates, variants, or analogs of these compounds in the lab (see, e.g., Gustafson, GR, Powers, DG, and Wuonola, MA. Use of 2-deoxystreptamine as a molecular scaffold for the preparation of functionally and spatially diverse molecules and pharmaceutical compositions thereof. Scriptgen Pharmaceuticals, Inc. 273964[6140361]. 10-31-2000. Waltham, MA. 3-22-1999). Many of these derivatives, conjugates, etc. also have been shown to have similar antiviral activity when compared to the parent compounds, and therefore, this invention encompasses the use of all lab-created derivatives, including but not limited to those compounds that involve aminoglycoside molecules conjugated to amino acids like arginine (e.g., Catani, MV, Corasaniti, MT, Ranalli, M, Amantea, D, Litovchick, A, Lapidot, A, and Melino, G (2003) The Tat antagonist neomycin B hexa-arginine conjugate inhibits gp-120-induced death of human neuroblastoma cells. J Neurochem 84[6]:1237-1245; Litovchick, A, Evdokimov, AG, and Lapidot, A (2000) Aminoglycoside-arginine conjugates that bind TAR RNA: synthesis, characterization, and antiviral activity. Biochem 39[11]:2838-2852; Litovchick, A, Lapidot, A, Eisenstein, M, Kalinkovich, A, and Borkow, G (2001) Neomycin B-arginine conjugate, a novel HIV-1 Tat antagonist: synthesis and anti-HIV activities. Biochem 40[51]:15612-15623). Indeed, it is possible that scientists will be able to envision and create several more derivatives, conjugates, and analogs of aminoglycoside molecules that also may be used in this invention to treat or prevent envelope virus infections. Further, certain other molecules such as viomycin (which is a cyclic peptide containing amino acids including arginine, serine, and lysine, and is a member of the tuberactinomycin family) and hygromycin B (which has an N3-methyl-2-deoxy-D-streptamine core) also are included in this invention as certain of their structures would permit them to have similar antiviral activity against envelope virus infection and protein translation (see, e.g., Wurmbach, P and Nierhaus, KH (1983) The inhibition pattern of antibiotics on the extent and accuracy of tRNA binding to the ribosome, and their effect on the subsequent steps in chain elongation. Eur J Biochem 130[1]:9-12).

Aminoglycoside (and structurally related) molecules have been shown to have inhibitory effects both on envelope virus attachment to the host cell membrane, and on translation of envelope virus proteins (and therefore on the proliferation rate of the envelope virus itself) (see, e.g., Langeland, N, Haarr, L, and Holmsen, H (1986) Evidence that neomycin inhibits HSV 1 infection of BHK cells. Biochem Biophys Res Commun 141[4]:198-203; Langeland, N, Oyan, AM, Marsden, HS, Cross, A, Glorioso, JC, Moore, LJ, and Haarr, L (1990) Localization on the herpes simplex virus type 1 genome of a region encoding proteins involved in adsorption to the cellular receptor. J Virol 64[3]:1271-1277; Zapp, ML, Stern, S, and Green, MR (1993) Small molecules that selectively block RNA binding of HIV-1 Rev protein inhibit Rev function and viral production. Cell 74[6]:969-978; Werstuck, G and Green, MR (1998) Controlling Gene Expression in Living Cells Through Small Molecule-RNA Interactions. Science 282[5387]:296-298). The latter effect in particular is thought to stem from the same reasons that these molecules are effective anti-bacterial agents: namely, their ability to bind to nucleic acids and effectively truncate protein translation by inducing misreading of viral codons.

It appears that aminoglycoside molecules, as well as their derivatives, analogs, etc., have the ability to recognize and bind to certain structural elements in ribosomal RNA that are characterized by non-Watson-and-Crick base-pairing, including hairpin loops, internal loops, bulges, or hexaloops. The binding of these molecules then interferes with the ability of the ribosome to translate the viral mRNA into proteins by inducing misreading of the codons in the viral mRNA (see, e.g., Werstuck, G and Green, MR (1998) Controlling Gene Expression in Living Cells Through Small Molecule-RNA Interactions. Science 282[5387]:296-298; Zapp, ML, Stern, S, and Green, MR (1993) Small molecules that selectively block RNA binding of HIV-1 Rev protein inhibit Rev function and viral production. Cell 74[6]:969-978). In addition, there is some evidence that these compounds can inhibit the activity of certain ribozymes, resulting in possible inhibition of group I intron self-splicing (von Ahsen, U and Schroeder, R (1991) Streptomycin inhibits splicing of group I introns by competition with the guanosine substrate. Nucleic Acids Res 19[9]:2261-2265), self-cleavage of the hammerhead (Stage, TK, Hertel, KJ, and Uhlenbeck, OC (1995) Inhibition of the hammerhead ribozyme by neomycin. RNA 1[1]:95-101), magnesium-induced self-cleavage reaction of the hairpin ribozyme (Earnshaw, DJ and Gait, MJ (1998) Hairpin ribozyme cleavage catalyzed by aminoglycoside antibiotics and the polyamine spermine in the absence of metal ions. Nucleic Acids Research 26[24]:5551-5561), and the tRNA processing activity of RNaseP RNA (Mikkelsen, NE, Brännvall M, Virtanen, A, and Kirsebom, LA (1999) Inhibition of RNase P RNA cleavage by aminoglycosides. Proc Natl Acad Sci U S A 96[11]:6155-6160). This invention utilizes these compounds to disrupt viral protein synthesis to decrease the overall viral titer of an infected individual by administering these compounds. Thus, in one embodiment of the present invention, disrupted viral protein synthesis results in the decreased ability of the viruses to replicate, spread, and cause clinically cognizable symptoms.

Aminoglycoside molecules and their relatives similarly have been shown to inhibit the binding of envelope viruses to host cells (see, e.g., Langeland, N, Haarr, L, and Holmsen, H (1986) Evidence that neomycin inhibits HSV 1 infection of BHK cells. Biochem Biophys Res Commun 141[4]:198-203; Langeland, N, Oyan, AM, Marsden, HS, Cross, A, Glorioso, JC, Moore, LJ, and Haarr, L (1990) Localization on the herpes simplex virus type 1 genome of a region encoding proteins involved in adsorption to the cellular receptor. J Virol 64[3]:1271-1277). These antiviral compounds purportedly bind to certain viral glycoproteins, which then effectively blocks the virus from attaching to and entering the host cell. The present invention could be used to disable the binding capabilities of viral glycoproteins, thereby preventing envelope viruses from initially infecting -- attaching to and penetrating – cells that were originally uninfected.

### ANTIVIRAL COMPOUND SYNTHESIS AND METHODS OF USE

An organic molecule capable of binding to RNA and inhibiting viral protein synthesis (in particular, protein translation) can be used to treat and prevent envelope virus infections in multicellular organisms. Both the proliferation and the cellular invasion of envelope viruses (which, for example, may include one or more of herpes simplex virus type 1, herpes simplex virus type 2, varicella zoster virus, toga virus, syncytial virus, paramyxovirus, myxovirus, human herpes virus-6, human immunodeficiency virus (HIV), cytomegalovirus, corona virus, or any of hepatitis A, B, C, D, E, or G) in multicellular organisms (such as, for example, humans or animals) may be halted or slowed by the administration of a compound whose active ingredient is an organic molecule capable of binding to RNA and inhibiting viral protein synthesis. Such an organic molecule may include, for example, a streptamine-containing aminoglycoside molecule (e.g., streptamycin), a spectinamine-containing aminoglycoside molecule (e.g., spectinomycin), a 2-DosA molecule (e.g., amikacin, butirosin, geneticin, gentamicin C1, gentamicin C1a, gentamicin C2, lividomycin, lividomycin A, neamine, neomycin, neomycin B, netilmicin, paromomycin, ribostamycin, sisomycin, tobramycin), a molecule in the tuberactinomycin family (e.g., viomycin), or molecules like hygromycin B, which contain a N3-methyl-2-deoxy-D-streptamine core. It is proposed that the overall titer of envelope viruses in the infected organism may be decreased as a result of the administration of said organic molecule, thereby decreasing the severity of the symptoms experienced by the infected organism.

Depending on the particular envelope virus infection being treated and the host organism to whom the treatment is being administered, the RNA-binding organic molecule is administered in its pure form, or as an active ingredient in a compound, comprising or consisting of one or more additives like an anti-microbial agent (e.g., polymyxin B, tetracycline), antiviral agent (e.g., cytokine), anti-fungal agent (e.g., micatin, tolnaftate), analgesic (e.g., pramoxine HCl), antioxidant (e.g., vitamin E), buffering agent, sunscreen (e.g., paraminobenzoic acid), cosmetic agent, fragrance, lubricant (e.g., synthetic or natural beeswax), oil, moisturizer, alcohol, drying agent (e.g., phenol, benzyl alcohol), preservative (e.g., methylparaben, propylparaben, benzyl alcohol, ethylene diamine tertraacetate salts), emulsifier, thickening agent (e.g., pullulin, xanthan, polyvinylpyrrolidone, carboxymethylcellulose), detergent (e.g., polyoxyl stearate, sodium lauryl sulfate), plasticizer (e.g., glycerol, polyethylene glycol), penetration enhancer (e.g., dimethylsulfoxide, phenol, isopropyl myristate, azone, salicylic acid, urea), or water, and be incorporated into a pharmaceutically acceptable carrier (e.g., cream, gel, lotion, ointment, suspension, aerosol spray, semi-solid formulation, suppository, liquid, solid, powder, or vapor). The compound can be administered in any manner that is physiologically appropriate with respect to both the ingredients of the compound (i.e. the particular organic molecule being used to bind to RNA and inhibit viral protein translation and any additives being used), the pharmaceutically acceptable carrier in which it is incorporated, and the envelope virus infection (or its symptoms) being treated. These physiologically appropriate manners include, but are not limited to, applying the compound topically, orally, sublingually, mucosally, trans-membranously, intravenously, intramuscularly, buccally, parentarelly, vaginally, anally, or transdermally.

While these compounds may be used for treatment or prevention of any envelope virus infection, the present invention is particularly well-suited for the treatment and prevention of cutaneous lesions caused by envelope viruses like herpes simplex-1 and varicella zoster, as shown in the following non-limiting examples.

### EXAMPLES

The examples of additives, RNA-binding molecules, pharmaceutically acceptable carriers, and physiologically appropriate manners of administration may sometimes be applicable to one or more of the described embodiments of the invention. The descriptions and examples provided in this application (including those in other sections), however, are explanatory and exemplary only, and should not be construed to restrict the invention as claimed.

Example 1: One embodiment of the invention is a composition comprising or consisting of an RNA-binding molecule and/or additives in a pharmaceutically acceptable carrier, which is administered to a human or animal in a physiologically appropriate manner in order to lower the titer of envelope viruses in said organism and prevent or ameliorate the occurrence of infection-related symptoms in the skin, eye, and mucous membranes of the organism. The composition may or may not be sterile, and the additives may include components that have physiological effects (e.g., one or more analgesics) so long as those effects do not interfere with the efficacy of the active ingredient (e.g., the RNA-binding molecule, etc.).

The carrier in this embodiment is preferably one suited to topical, transmembranous, ocular, or mucosal administration – for example, a carrier comprising or consisting of: an aqueous or oleaginous base (e.g., one or more of white petrolatum, isopropyl myristate, lanolin or lanolin alcohols, mineral oil, sorbitan mono-oleate, propylene glycol, cetylstearyl alcohol), a detergent (e.g., polyoxyl stearate or sodium lauryl sulfate), and water, thus forming a lotion, cream, or semi-solid carrier. Other suitable carriers may comprise of mixtures of emulsifiers and emollients, sucrose cocoate, sucrose distearate, mineral oil, propylene glycol, 2-ethyl-1,3-hexanediol, polyoxypropylene-15-stearyl ether and water.

Dilute suspensions without thickeners are most suitable for delivery to skin surfaces as aerosol sprays. For delivery to ocular surfaces, a base of a sterile saline solution is recommended.

The composition may contain additives the composer chooses. Preferably, such additives (e.g., sunscreens, moisturizers, etc.) do not affect the efficacy of the active ingredient. Particularly where the manner of administration is either topical or transmembranous, one of the additives may be a penetration enhancer (e.g., dimethylsulfoxide, a phenol, etc.).

In an embodiment of the present invention, administration of the compound comprises 1 mg to 10 mg of the compound (which, as prepared with additives in a pharmaceutically acceptable carrier, contains 0.1-40% by weight of the RNA-binding molecule as the active ingredient), administered once every three to twelve hours, for one to fourteen days. Most preferably, the compound is administered two to six times per day, using 0.1 to 5g per application (of 0.1-20% by weight of the active ingredient), for one to seven days. For administrations used to combat physiological symptoms of infection, it is preferable to begin applying the compound to the affected tissues as soon as the first symptoms are detected.

Example 2: This embodiment of the invention is a compound similar to that disclosed in Example 1, except that it is specifically targeted to the treatment of herpes simplex I and the prevention of physiological manifestations of herpes simplex I (e.g., cold sores).

In one ultimately effective experiment, between 3.5 and 5 mg/g of neomycin B was used, combined with pramoxine hydrochloride (for pain relief) in petroleum jelly. However, for longer-term use or storage, preservatives like methylparaben are utilized to avoid contamination, and the ingredients preferably comprise an emulsion or suspension to distribute evenly the physiologically active ingredients.

Administration of the compound consists of 1 mg to 10 mg of the compound (which, as prepared with additives in a pharmaceutically acceptable carrier, contains 0.1-40% by weight of the RNA-binding molecule as the active ingredient), administered once every three to twelve hours, for one to fourteen days. Most preferably, the compound is administered two to six times per day, using 0.1 to 5g per application (of 0.1-20% by weight of the active ingredient); one to seven days of treatment may be sufficient to treat an existing herpes-related lesion or prevent one from erupting or progressing. Apply the compound topically to the affected skin as soon as skin-related symptoms (e.g., inflammation, redness, tingling, swelling, itching, burning, cracking, blistering, bleeding, oozing, and/ or weeping) are detected.

Example 3: In one embodiment of the invention, a compound comprising or consisting of at least one 2-DosA molecule is administered to at least one of the skin, eye, cornea, and/or mucous membrane of a human or animal to treat or prevent varicella zoster virus infection. The at least one 2-Dos A molecule (which can be any of amikacin, butirosin, geneticin, gentamicin C1, gentamicin C1a, gentamicin C2, lividomycin, lividomycin A, neamine, neomycin, neomycin B, netilmicin, paromomycin, ribostamycin, sisomycin, or tobramycin) is combined with additives (in this example, the most appropriate additives may include: water, non-irritating organic molecules, and physiologically active additives like analgesics if they do not interfere with the efficacy of the 2-DosA molecules) in a pharmaceutically acceptable carrier that is physiologically compatible with the targeted tissues to which the compound is administered– skin, eye, and mucous membrane tissues. In this example, such carriers include a cream, gel, lotion, ointment, aerosol spray or semi-solid (e.g., suppository) formulation, and any additives are inactive, so as to make a suspension of the active ingredient or ingredients.

The carrier in this embodiment is preferably one suited to topical, transmembranous, ocular, or mucosal administration – for example, a carrier comprising or consisting of: an aqueous or oleaginous base (e.g., one or more of white petrolatum, isopropyl myristate, lanolin or lanolin alcohols, mineral oil, sorbitan mono-oleate, propylene glycol, cetylstearyl alcohol), a detergent (e.g., polyoxyl stearate or sodium lauryl sulfate), and water, thus forming a lotion, cream, or semi-solid carrier. Other suitable carriers may comprise of mixtures of emulsifiers and emollients, sucrose cocoate, sucrose distearate, mineral oil, propylene glycol, 2-ethyl-1,3-hexanediol, polyoxypropylene-15-stearyl ether and water.

The composition may contain any additives the composer chooses, so long as such additives (e.g., sunscreens, moisturizers, etc.) do not affect the efficacy of the active ingredient. Particularly where the manner of administration is either topical or transmembranous, one of the additives may be a penetration enhancer (e.g., dimethylsulfoxide, a phenol, etc.).

In an embodiment of the present invention, administration of the compound comprises 1 mg to 10 mg of the compound (which, as prepared with additives in a pharmaceutically acceptable carrier, contains 0.1-40% by weight of the RNA-binding molecule as the active ingredient), administered once every three to twelve hours, for one to fourteen days. Most preferably, the compound is administered two to six times per day, using 0.1 to 5g per application (of 0.1-20% by weight of the active ingredient), for one to seven days. For administrations used to combat physiological symptoms of infection, it is preferable to begin applying the compound to the affected tissues as soon as the first symptoms of the varicella zoster virus are detected.

Example 4: This embodiment of the invention provides a method for administering a compound comprising 2-DosA in a pharmaceutically acceptable carrier to humans or animals either intravenously or intramuscularly. The composition of the carrier may vary, so long as the carrier is compatible with the blood and tissues of the human or animal such that it may be injected into these tissues without causing physiological effects that may be either deleterious to the human or animal or interfere with the function of the active ingredient.

This composition comprises 2-DosA in concentrations from 0.01 mg/ml to 500 mg/ml and suspended in a carrier solution of isotonic sodium chloride solution containing a suitable preservative, such as 0.1 to 1.5% benzyl alcohol, stabilizers such as from 0.25 to 1% carboxymethylcellulose sodium and 0.005 to 0.1% polysorbate 80, and sufficient sodium hydroxide or hydrochloric acid to adjust the pH to 5.0 to 7.5 (all percentages by weight). This compound may be used for either intravenous or intramuscular injection. Another embodiment of the invention suitable for either intramuscular or intravenous injection is a composition comprising at least one 2-DosA molecule in concentrations from 0.01 mg/ml to 500 mg/ml and suspended in a carrier solution of alcohol (1-10%), glycerin (10-20%) and water (balance 70-89%), along with a suitable preservative (all percentages by weight).

Such compositions may be injected in suitable amounts to provide a dose to the patient of from 0.1 mg/50 kg body weight to 2 gm/50 kg body weight. It is desirable to achieve and maintain a level of the specified alcohol(s) in the body in the range of at least about 0.1 mg/kg of body weight.

The alcohol(s) to which this invention is directed may effectively be introduced through the mucus membrane system of the human or animal patient. Such introduction may be, for example, through the vaginal, anal, or nasal membranes. The above liquid compositions, which comprise of one or more aliphatic alcohols, having from 27 to 32 carbons in the aliphatic chain of such alcohol(s), in a suitable liquid carrier may, for example, be used for trans-mucus-membranal introduction of such alcohol(s) into the circulatory system of the human or mammal to be treated by, for example, introducing such liquid as an aerosol into the oral or nasal passages or as liquid into the vaginal or anal passages of the body where these compounds inactivate the envelope virus locally, inhibit the passage of the envelope virus into the cell membrane, and pass through the cell membrane into the circulatory system of the patient where the compounds act as inhibitors of viral activity and infectivity and inactivate virus. In the latter applications, however, gels, creams or suppositories are more conveniently used.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. A composition comprising: at least one organic molecule that binds to RNA and inhibits translation of envelope virus proteins; and a pharmaceutically acceptable carrier.

2. The composition of claim 1, wherein the envelope virus comprises herpes simplex virus type 1, herpes simplex virus type 2, varicella zoster virus, toga virus, syncytial virus, paramyxovirus, myxovirus, human herpes virus-6, human immunodeficiency virus (HIV), cytomegalovirus, hepatitis A, hepatitis B, hepatitis C, hepatitis D, hepatitis E, hepatitis G, corona virus, or a combination thereof.

3. The composition of claim 1, wherein the at least one organic molecule comprises at least one 2-deoxystreptamine aminoglycoside molecule.

4. The composition of claim 1, wherein the at least one organic molecule comprises at least one conjugate of a 2-deoxystreptamine aminoglycoside molecule.

5. The composition of claim 3, wherein the at least one 2-deoxystreptamine aminoglycoside molecule comprises amikacin, butirosin, geneticin, gentamicin C1, gentamicin C1a, gentamicin C2, lividomycin, lividomycin A, neamine, neomycin, neomycin B, netilmicin, paromomycin, ribostamycin, sisomycin, tobramycin, or a combination thereof.

6. The composition of claim 1, wherein the at least one organic molecule comprises tuberactinomycin.

7. The composition of claim 6, wherein the at least one organic molecule comprises viomycin.

8. The composition of claim 1, wherein the at least one organic molecule comprises a 1,3-diaminocyclohexane structure.

9. The composition of claim 8, wherein the at least one organic molecule comprises spectinomycin.

10. The composition of claim 8, wherein the at least one organic molecule comprises hygromycin B.

11. The composition of claim 8, wherein the at least one organic molecule comprises streptomycin.

12. The composition of claim 1, wherein the at least one organic molecule comprises at least one analog of a 2-deoxystreptamine aminoglycoside molecule.

13. The composition of claim 12, wherein the at least one analog of a 2-deoxystreptamine aminoglycoside molecule is functionalized with at least one modifying group.

14. The composition of claim 1, wherein the composition further comprises a penetration enhancer.

15. The composition of claim 1, wherein said pharmaceutically acceptable carrier comprises cream, gel, lotion, ointment, suspension, aerosol spray, semi-solid formulation, suppository, liquid, solid, powder, vapor, or a combination thereof.

16. The composition of claim 1, wherein the at least one organic molecule is present a concentration from 0.01% to 40%, by weight.

17. The composition of claim 1, wherein the composition further comprises an additive.

18. The composition of claim 17, wherein the additive comprises an anti-microbial agent, antiviral agent, anti-fungal agent, analgesic, antioxidant, buffering agent, sunscreen, cosmetic agent, fragrance, lubricant, oil, moisturizer, alcohol, drying agent, preservative, emulsifier, thickening agent, detergent, plasticizer, penetration enhancer, water, or a combination thereof.

19. The composition of claim 3, wherein the at least one 2-deoxystreptamine aminoglycoside molecule comprises from one to five sugar moieties.

20. The composition of claim 3, wherein at least one hydroxyl group of the at least one 2-deoxystreptamine aminoglycoside molecule is functionalized with modifying groups.

21. The composition of claim 3, wherein at least one amino group of the at least one 2-deoxystreptamine aminoglycoside molecule is functionalized with at least one modifying group.

22. The composition of claim 21, wherein the at least one modifying group is arginine.

23. The composition of claim 3, wherein at least one amino group of the at least one 2-deoxystreptamine aminoglycoside molecule is a primary amine.

24. The composition of claim 3, wherein at least one amino group of the at least one 2-deoxystreptamine aminoglycoside molecule is methyl substituted.

25. The composition of claim 3, wherein none of the hydroxyl moieties of the at least one 2-deoxystreptamine aminoglycoside molecule are functionalized.

26. The composition of claim 1, wherein the pharmaceutically acceptable carrier does not contain an organic molecule that binds to RNA and inhibits translation of envelope virus proteins.

27. The composition of claim 1, wherein said at least one organic molecule comprises Structure I: 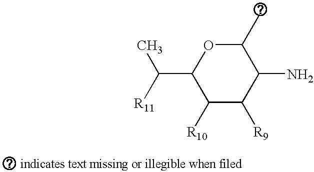
, wherein R₁ comprises H, CH₂CH₃, or L-(-)-4-amino-2-hydroxybutyrl; wherein R₂ comprises 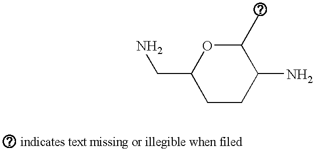
, 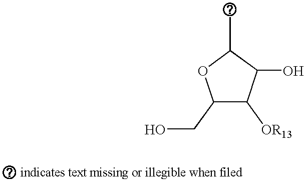
, or 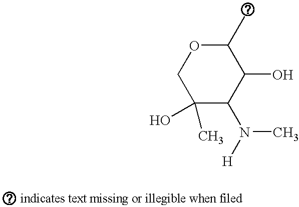
; wherein R₅ comprises NH₂, OH, or arginine; wherein R₆ comprises H or OH; wherein R₇ comprises H or OH; wherein R₈ comprises NH₂, OH, or arginine; wherein R₉ comprises H or OH; wherein R₁₀ comprises H or OH; wherein R₁₁ comprises NH₂, OH, NHCH₃, or arginine; wherein R₃ comprises H, 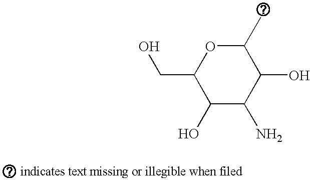
, 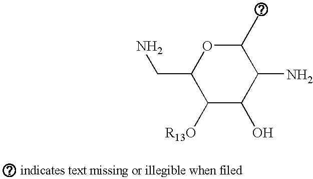
, 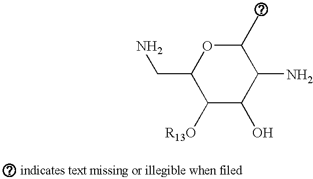
or 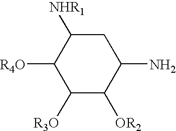
; wherein R₁₂ comprises H or 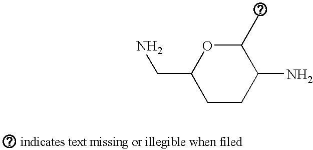
; wherein R₁₃ comprises H, mannosyl, 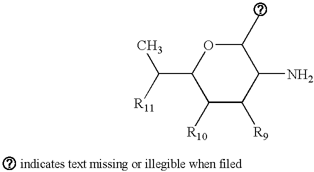
, 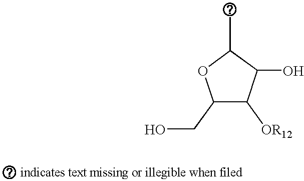
, or 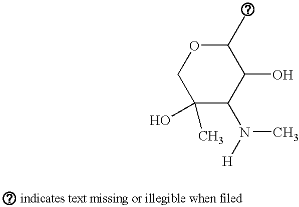
; and wherein R₄ comprises H, 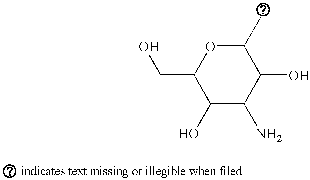
, 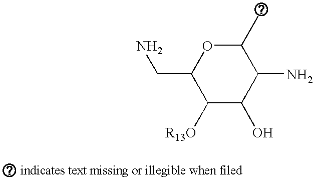
, 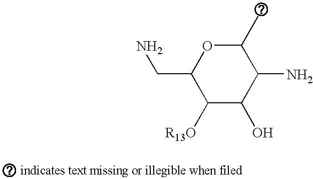
, or 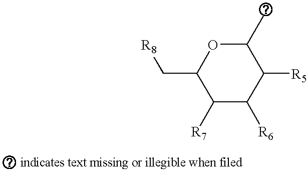
.

28. The composition of claim 27, wherein R₃ and R₄ independently comprise H.

29. The composition of claim 27, wherein at least one amino group in said 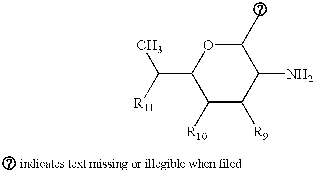
, is substituted with at least one arginine.

30. The composition of claim 27, wherein the amino group in said 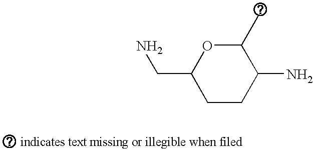
, is substituted with at least one arginine.

31. The composition of claim 27, wherein the NHR₁ in said 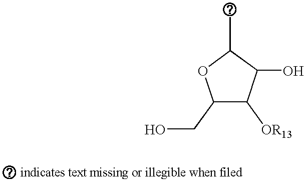
, is substituted with at least one arginine.

32. A method of treating envelope virus infections in multicellular organisms, comprising the steps of: (a) administering a composition in a physiologically appropriate manner to the organism infected with an envelope virus; wherein the composition is administered at least one time; wherein the composition comprises at least one organic molecule that binds to RNA and inhibits translation of envelope virus protein and a pharmaceutically acceptable carrier.

33. The method of claim 32, wherein the at least one organic molecule is present in a concentration from about 0.01% to about 40%, by weight.

34. The method of claim 32, wherein the composition is administered to the organism at least one time per day.

35. The method of claim 32, wherein the composition is administered to the organism over a time period comprising at least one day.

36. The method of claim 32, wherein said method further comprises prior to step (a), diagnosing clinical symptoms of the envelope virus in the organism.

37. The method of claim 36, wherein said clinical symptoms comprise detectable presence of viral antibodies in body fluids, diagnostic levels of viral titer in body fluids, pain, swelling, burning, inflammation, redness, tingling, itching, skin lesions, or a combination thereof.

38. The method of claim 32, wherein the pharmaceutically acceptable carrier comprises cream, gel, lotion, ointment, suspension, aerosol spray, semi-solid formulation, suppository, liquid, solid, powder, vapor, or a combination thereof.

39. The method of claim 32, wherein the pharmaceutically acceptable carrier further comprises at least one additive.

40. The method of claim 39, wherein the at least one additive comprises an anti-microbial agent, antiviral agent, anti-fungal agent, analgesic, antioxidant, buffering agent, sunscreen, cosmetic agent, fragrance, lubricant, oil, moisturizer, alcohol, drying agent, preservative, emulsifier, thickening agent, detergent, plasticizer, penetration enhancer, and water.

41. The method of claim 32, wherein the multicellular organism is a human.

42. The method of claim 32, wherein the pharmaceutically acceptable carrier does not contain an organic molecule that binds to RNA and inhibits translation of envelope virus proteins.

43. The method of claim 32, wherein said step administering said composition in a physiologically appropriate manner comprises administering the composition topically, orally, sublingually, mucosally, trans-membranously, intravenously, intramuscularly, buccally, parentarelly, vaginally, anally, transdermally, or a combination thereof.

44. The method of claim 32, wherein the at least one organic molecule comprises at least one 2-deoxystreptamine aminoglycoside molecule.

45. The method of claim 32, wherein the at least one organic molecule comprises at least one conjugate of a 2-deoxystreptamine aminoglycoside molecule.

46. The method of claim 44, wherein the at least one 2-deoxystreptamine aminoglycoside molecule comprises amikacin, butirosin, geneticin, gentamicin C1, gentamicin C1a, gentamicin C2, lividomycin, lividomycin A, neamine, neomycin, neomycin B, netilmicin, paromomycin, ribostamycin, sisomycin, tobramycin, or a combination thereof.

47. The method of claim 32, wherein the at least one organic molecule comprises tuberactinomycin.

48. The method of claim 47, wherein the at least one organic molecule comprises viomycin.

49. The method of claim 32, wherein the at least one organic molecule comprises a 1,3-diaminocyclohexane structure.

50. The method of claim 32, wherein the at least one organic molecule comprises spectinomycin.

51. The method of claim 32, wherein the at least one organic molecule comprises hygromycin B.

52. The method of claim 32, wherein the at least one organic molecule comprises streptomycin.

53. The method of claim 32, wherein the at least one organic molecule comprises at least one analog of a 2-deoxystreptamine aminoglycoside molecule.

54. The method of claim 53, wherein the at least one analog of a 2-deoxystreptamine aminoglycoside molecule is functionalized with at least one modifying group.

55. A method of treating skin lesions caused by at least one envelope virus in multicellular organisms, said method comprising: (a) administering a composition in a physiologically appropriate manner; wherein the composition is administered at least one time; wherein the composition comprises at least one organic molecule that binds to RNA and inhibits translation of envelope virus protein and a pharmaceutically acceptable carrier.

56. The method of claim 55, wherein the at least one organic molecule comprises at least one 2-deoxystreptamine aminoglycoside molecule.

57. The method of claim 55, wherein the at least one organic molecule comprises at least one conjugate of a 2-deoxystreptamine aminoglycoside molecule.

58. The method of claim 56, wherein the at least one 2-deoxystreptamine aminoglycoside molecule comprises amikacin, butirosin, geneticin, gentamicin C1, gentamicin C1a, gentamicin C2, lividomycin, lividomycin A, neamine, neomycin, neomycin B, netilmicin, paromomycin, ribostamycin, sisomycin, tobramycin, or a combination thereof.

59. The method of claim 55, wherein the at least one organic molecule comprises tuberactinomycin.

60. The method of claim 55, wherein the at least one organic molecule comprises viomycin.

61. The method of claim 55, wherein the at least one organic molecule comprises a 1,3-diaminocyclohexane structure.

62. The method of claim 61, wherein the at least one organic molecule comprises spectinomycin.

63. The method of claim 61, wherein the at least one organic molecule comprises hygromycin B.

64. The method of claim 61, wherein the at least one organic molecule comprises streptomycin.

65. The method of claim 55, wherein the at least one organic molecule comprises at least one analog of a 2-deoxystreptamine aminoglycoside molecule.

66. The method of claim 65, wherein the at least one analog of a 2-deoxystreptamine aminoglycoside molecule is functionalized with at least one modifying group.

67. The method of claim 55, wherein the at least one organic molecule is present in a concentration from about 0.01% to about 40%, by weight.

68. The method of claim 55, wherein the composition is administered to the organism at least one time per day.

69. The method of claim 55, wherein the composition is administered to the organism over a time period comprising at least one day.

70. The method of claim 55, wherein said method further comprises prior to step (a), diagnosing clinical symptoms of the envelope virus in the organism.

71. The method of claim 70, wherein said clinical symptoms comprise skin-related symptoms.

72. The method of claim 71, wherein said skin-related symptoms comprise inflammation, redness, tingling, swelling, itching, burning, cracking, blistering, bleeding, oozing, weeping, or a combination thereof.

73. The method of claim 55, wherein the pharmaceutically acceptable carrier comprises cream, gel, lotion, ointment, suspension, aerosol spray, semi-solid formulation, suppository, liquid, solid, powder, vapor, or a combination thereof.

74. The method of claim 55, wherein the pharmaceutically acceptable carrier further comprises at least one additive.

75. The method of claim 74, wherein the at least one additive comprises an anti-microbial agent, antiviral agent, anti-fungal agent, analgesic, antioxidant, buffering agent, sunscreen, cosmetic agent, fragrance, lubricant, oil, moisturizer, alcohol, drying agent, preservative, emulsifier, thickening agent, detergent, plasticizer, penetration enhancer, and water.

76. The method of claim 55, wherein the multicellular organism is a human.

77. The method of claim 55, wherein the pharmaceutically acceptable carrier does not contain an organic molecule that binds to RNA and inhibits translation of envelope virus proteins.

78. The method of claim 55, wherein said step administering said composition in a physiologically appropriate manner comprises administering the composition topically, orally, sublingually, mucosally, trans-membranously, buccally, parentarelly, vaginally, anally, transdermally, or a combination thereof.
